(19) United States
(12) Patent Application Publication
(10) Pub. No.: US 2026/0207999 A1
(43) Pub. Date: Jul. 23, 2026

(54) SYSTEMS AND METHODS FOR MOTIVATING AND DRIVING FITNESS AND EXERCISE ROUTINES

(71) Applicant: Towers Fantasy World, Inc., Holbrook, NY (US)

(72) Inventor: Matthew Towers, Holbrook, NY (US)

(21) Appl. No.: 19/454,901

(22) Filed: Jan. 21, 2026

Related U.S. Application Data
(60) Provisional application No. 63/747,618, filed on Jan. 21, 2025.

(51) Int. Cl. A63B 24/00

(52) U.S. Cl. CPC A63B 24/0087; A63B 24/0062; A63B 2024/0065; A63B 2024/0068; A63B 2024/0096; A63B 2220/30; A63B 2220/40; A63B 2220/836; A63B 2225/50; A63B 2230/06

(57) ABSTRACT

Disclosed are systems and methods for motivating and driving fitness and exercise routines that include at least one server communicatively coupled to at least one data storage device configured to store bio data, vital sign data, movement data, fitness data, aggregated data, and skills data, and at least one fitness device utilized by a user engaged in a fitness routine, wherein the fitness device generates data based on data produced by the user during the fitness routine and communicates the at least one of the vital sign data and the movement data to the server. The system receives incomplete and heterogeneous biometric data from different devices, maps that data into a skill space, compensates missing parameters, and equalizes users so that differences in sensor coverage do not produce unfair advantages. The results can then be exposed through an API for use by external games and applications.

### TECHNICAL FIELD

The systems and methods disclosed herein relate to enhancing a user experience in an interactive application (app) based on previous user fitness and exercise routines.

### BACKGROUND

Many people struggle to stay motivated to exercise regularly, leading to wasted time, energy, and potential health benefits. In addition, many people who start an exercise program quit within the first month, wasting resources spent on gym memberships and fitness equipment. Lack of motivation and engagement results in wasted potential health benefits and increased healthcare costs due to preventable illnesses.

In the prior art, fitness data was used in a game or other application to provide the user with extra points for game play. These prior art systems used a linear approach to this enhanced game play, for example, if a user walked 20 steps, the user was awarded 20 game points. These prior art systems are limited in function and lack any real motivational aspects in modern day applications.

There is a longstanding need for a system that motivates and drives a user to engage in long-term exercise and fitness routines.

### SUMMARY OF THE INVENTION

The systems and methods disclosed herein relate to enhancing a user experience in an interactive application (app) based on previous user fitness and exercise routines.

An aspect of the disclosure is a system for motivating and driving fitness and exercise routines. The system includes at least one server communicatively coupled to, at least one data storage device configured to store bio data, vital sign data, movement data, fitness data, aggregated data, and skills data; and at least one fitness device utilized by a user engaged in a fitness routine, wherein said fitness device generates at least one of the vital sign data and the movement data produced by the user during the fitness routine and communicates the at least one of the vital sign data and the movement data to the server. The server receives the data and calculates skill data that can be used in third party apps or businesses.

### BRIEF DESCRIPTION OF THE DRAWINGS

The drawings described herein are for illustration purposes only. The drawings are not necessarily to scale, with emphasis generally being placed upon illustrating the principles of the present teachings. The drawings are not intended to limit the scope of the present teachings in any way. Like numerals generally refer to like parts.

FIG. 1 is a diagram of a system for motivating and driving fitness and exercise routines according to the present disclosure;

FIG. 2 is an example of a cell phone user application depicting a dashboard for viewing various user fitness data in a user summary mode according to the present disclosure;

FIG. 3 is an example of a cell phone user application depicting a dashboard for viewing various user fitness data in a fitness metric mode according to the present disclosure;

FIG. 4 is an example of a cell phone user application depicting an interface to initiate a connection to various input devices according to the present disclosure;

FIG. 5 is an example of a cell phone user application depicting an interface to initiate a data transfer to third party applications according to the present disclosure;

FIG. 6 is a diagram of a server according to the present disclosure;

FIG. 7 is a flow chart illustrating a method for motivating and driving fitness and exercise routines according to the present disclosure; and

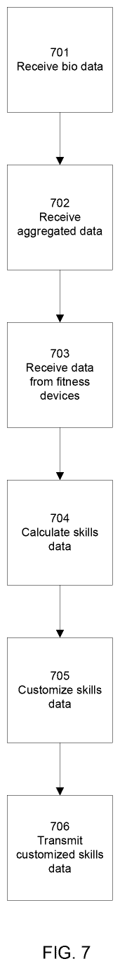

FIG. 8 is a flowchart illustrating the equalization and normalization processes according to the present disclosure.

Like reference numerals indicate similar parts throughout the figures.

### DETAILED DESCRIPTION

It is to be understood that the invention herein is not limited to the particular procedures, materials and modifications described herein and as such may vary. It is also to be understood that the terminology used is for purposes of describing particular embodiments only and is not intended to limit the scope of the present invention which will be limited only by the appended claims.

In the specification and claims herein, the following terms and expressions are to be understood as indicated.

The singular forms “a,” “an,” and “the” include the plural.

All methods described herein may be performed in any suitable order unless otherwise indicated herein or otherwise clearly contradicted by context. The use of any and all examples, or exemplary language provided herein, e.g., “such as”, is intended merely to better illuminate the invention and does not pose a limitation on the scope of the invention unless otherwise claimed. No language in the specification should be construed as indicating any non-claimed element as essential to the practice of the invention.

As used herein, “comprising,” including,” “containing,” “characterized by”, and grammatical equivalents thereof are inclusive or open-ended terms that do not exclude additional, unrecited elements or method steps, but will also be understood to include the more restrictive terms “consisting of” and “consisting essentially of.”

Other than in the working examples or where otherwise indicated, all numbers expressing amounts of materials, reaction conditions, time durations, quantified properties of materials, and so forth, stated in the specification and claims are to be understood as being modified in all instances by the term “about.”

It will be understood that any numerical range recited herein includes all sub-ranges within that range and any combination of the various endpoints of such ranges or sub-ranges.

Disclosed herein is a fitness concept that can integrate augmented reality into sports exercises, offering users an interactive and engaging way to improve their fitness levels. The system is a platform that collects users' fitness data and uses it to create values in a table of proprietary skills using custom formula which is then incorporated into games and other applications. Those values evolve along with the user's real-life fitness journey. In general, the user's fitness and activity data is gathered with the user's wearable devices acting as sensors and the data Is transmitted to a remote server. The system can use a machine learning model to create custom values and skills that can be uploaded to any game or application. The system can integrate augmented reality into sports exercises, offering users an interactive and engaging way to improve their fitness levels.

An aspect of the invention incorporates the presence of an equalization and normalization layer inside the system. The system includes an equalizing algorithm that adjusts skill values when some aggregated data is missing, and this equalization can be driven by large language models. The resulting skill data can be customized and delivered to third party applications.

Disclosed is a physiological normalization middleware. This middleware receives incomplete and heterogeneous biometric data from different devices, maps the data into a canonical multi-dimensional skill space, infers or compensates missing physiological parameters, and equalizes users so that differences in sensor coverage do not produce unfair advantages. The normalized results are then exposed through an API for use by external games and applications.

This type of middleware does not exist in current commercial platforms. Existing health platforms and games consume raw or lightly processed fitness data, but they do not provide a normalized, cross device, fairness adjusted skill layer that can be directly consumed by third party applications. The disclosed system creates a mathematical and computational layer between the human body and external digital experiences, which is the true patentable subject matter.

Disclosed is a system wherein multiple users with radically different sensor coverage can still be compared and used in a fair and consistent manner. One user may only provide step data, while another may provide a full set of biometric measurements, yet the system produces comparable and balanced RPG style statistics for both users. No prior art platform provides this type of cross sensor, fairness adjusted normalization layer for external applications.

Disclosed is a system for generating game ready skill values from heterogeneous biometric sources, comprising a server configured to receive multiple physiological and activity datasets from heterogeneous fitness devices, a normalization engine that maps those datasets into a canonical multi-dimensional skill space, a missing data compensation engine that infers or weights absent physiological parameters based on available parameters and historical user models, a fairness equalization engine that normalizes the resulting skill values across users having different sensor coverage, and an API layer that exposes the normalized skill values to third party applications independently of the original biometric sources.

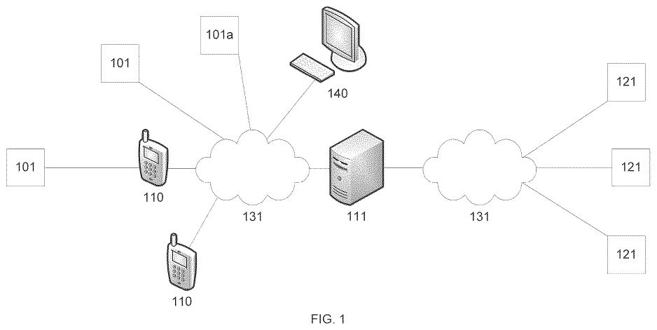

Referring to FIG. 1, illustrated is a system for motivating and driving fitness and exercise routines. The system generally includes one or more input devices 101, one or more networks 131, and at least one server 111. The system can also include a smart phone 110. Data generated by the at least one server 111 can be communicated to one or more user applications (“apps”) 121 for use therein, as will be described in further detail below.

The server is configured to receive multiple physiological and activity datasets from heterogeneous fitness devices. The server includes a normalization engine that maps those datasets into a canonical multi-dimensional skill space and a missing data compensation engine that infers or weights absent physiological parameters based on available parameters and historical user models. Also included is a fairness equalization engine that normalizes the resulting skill values across users having different sensor coverage. An API layer can expose the normalized skill values to third party applications independently of the original biometric sources.

An input device 101, e.g., fitness device, includes various types of data collection devices that collect vital sign data and/or movement data of a user. The input device can include smart watches, fitness watches, smart weights, smart scales, smart arm bands, smart ankle bands, heart monitors, smart phones, to name a few. An input device 101 also includes a power supply, e.g., a rechargeable battery. Vital sign data (or physiological data) can include, but is not limited to, heart rate, blood pressure, pulse, breathing rate, oxygen saturation, etc. Movement data (or activity data) can include, but is not limited to, distance, time, sensor type, direction of movement, etc.

The data generated by these input devices is eventually received by server 111 for processing. It is noted that data generated by the input devices 101 can be received and subsequently transmitted to the server 111 via a cell phone (or other smart device) 110. For example, if the input device is a smart watch, the smart watch can transmit the data to the smart phone 110, which in turn would transmit the data to the server 111 through network 131. In other embodiments, the data generated by the input devices is transmitted directly to the server 111 through the network 131. For example, if the input device is a smart device connected to the Internet of Things (IoT), for example a smart scale, the smart device can collect the data and transmit the data directly to the server 111 via the network 131. The input devices 101 can transmit the data via Bluetooth, Wi-Fi, or other wireless protocol. More than one type of fitness device can be utilized by the system to generate multiple sources of vital sign data and movement data.

In general, the input device 101 can be any type of fitness device that reads vital signs of a user and/or detects motion of a user, or both. It is noted that some input devices can provide calculated values such as speed (i.e., distance×time), but it is understood that the server 111 can also calculate these data points from the raw data received from the input devices 101.

Some examples of vital sign and motion include, but not limited to: steps of a user; distance traveled by walking, running, or cycling; flights climbed; workouts: specific workouts logs, such as running, cycling, swimming, yoga, or strength training, and track metrics like duration, intensity, distance, and calories burned; active calories; resting calories; exercise minutes; stand hours; heart rate, throughout the day and during exercise; VO2 max, as a measure of cardiorespiratory fitness; recovery: time after a workout, which is the time it takes for your heart rate to return to normal; workout route maps using GPS; sleep analysis: sleep duration and quality, including time spent asleep, time awake, and the different stages of sleep (light, deep, rem); activity tracking: steps, distance traveled, and calories burned throughout the day; exercise tracking: specific exercises, such as running, cycling, and swimming, and provides metrics like duration, distance, and calories burned; weight management: weight loss or gain goals and tracking over time; stress management stress tracking stress levels based on heart rate variability; weight and body composition: weight, body fat percentage, and BMI (Body Mass Index); health metrics: additional health metrics, such as oxygen saturation (SpO2), skin temperature, and menstrual health (for female users). Other vital data is user input. For example: nutrition tracking: track daily food intake, including calories consumed, macronutrient breakdown, and hydration levels.

Input devices 101 can include components such as gyroscopes, accelerometers, heart rate sensors, timers, GPS receivers, O2 sensors, thermometers, and the like to generate the vital sign data and/or movement data of the user. The input devices 101 can be positioned at different locations on a user, for example, as an arm unit, a wrist unit, a head unit, a chest unit, a leg unit, an ankle unit, etc. The different locations will produce different fitness data generated by the user during a workout or other fitness activity. As an example of how the input devices 101 operate, the following illustration is provided:
Step 1: The user turns on the input device—it is noted that the input devices may wake up from ‘sleep mode’ when they detect movement;
Step 2: The user puts the input devices on the user's body—e.g., 2 sensors on the arms, 2 sensors on the legs, and 1 sensor on the chest;
Step 3: The user performs the desired workout;
Step 4: Throughout the workout the input devices capture and store fitness data;
Step 5: The fitness data is transmitted to the phone app when it is opened, and nearby;
Step 6: The phone app translates the fitness data into skill data, which is then translated to the custom video game; and
Step 7: When the user is done, the input devices are turned off and removed—it is noted that the sensors may go into ‘sleep mode’ instead of completely turning off.

This process may be modified as needed to specific input device configuration and game requirements.

It is noted that fitness data can also be obtained from third party input devices 101a, apps or businesses, for example, data generated under a subscription for a cycle routine can be received by the system as fitness data for use in later calculations.

The input device 101 can include transmitters that transmit the data via one or more wireless protocols, for example, a Bluetooth Low Energy (BLE) module; other wired and/or wireless protocols are contemplated.

It is noted that the one or more network 111 can be a wired network, wireless network, or a combination of both. In addition, various wireless and wired protocols can be utilized to support the transmission of the various data.

Cell phone 110 includes a locally running user application (e.g., a dashboard) to assist the user with the input of data and display of data generated by the system. Cell phone 110 is used by the user to enter basic information such as name and bio data. The bio data can include the user's age, gender, height, weight, etc. The information and bio data is transmitted to the server 111 for storage and processing.

In addition, the cell phone 110 can have various graphical user interfaces (GUIs) to assist the user in accessing the server 111.

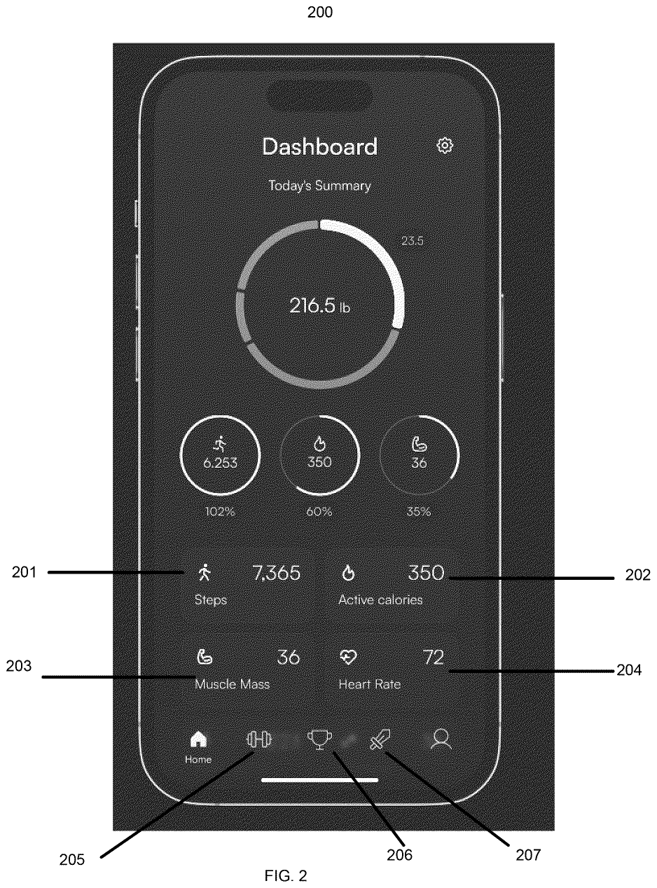

FIG. 2 is an example of a cell phone user application depicting a dashboard for viewing various user fitness data in a user summary mode. This particular GUI 200 provides the user with certain aggregated data, for example, steps 201, active calories 202, muscle mass 203, heart rate 204, BMI, workouts (barbell) 205, accomplishment rating (trophy) 206, gaming link (sword) 207, etc. The GUI is for example purposes, as one skilled in the art would understand other data and configurations of the display are possible.

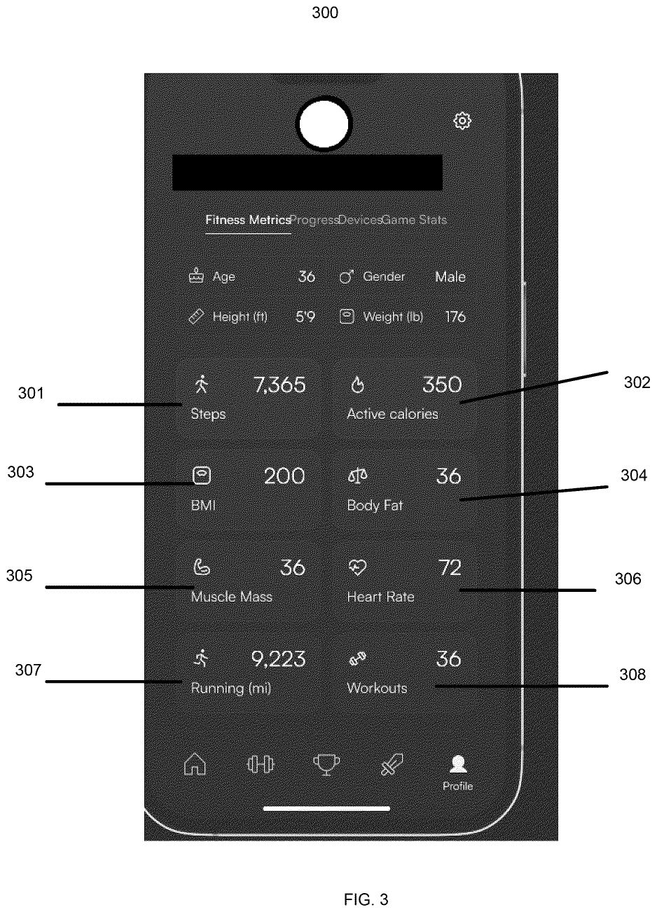

FIG. 3 is an example of a cell phone user application depicting a dashboard for viewing various user fitness data in a fitness metric mode. This particular GUI 300 provides the user with certain bio data (age, weight, gender, height, etc.) and aggregated data, for example, steps 301, active calories 302, muscle mass 305, heart rate 306, BMI 303, body fat 304, workouts (barbell) 308, running distance 307, etc. The GUI is for example purposes, as one skilled in the art would understand other data and configurations of the display are possible.

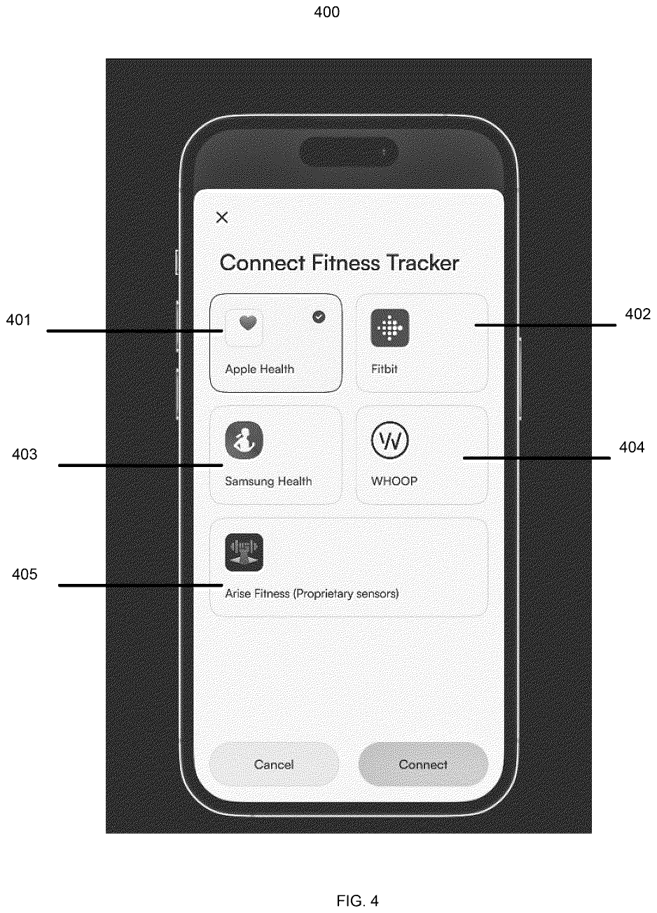

FIG. 4 is an example of a cell phone user application depicting an interface to initiate a connection to various input devices. This particular GUI 400 provides the user with the ability to connect and disconnect input devices, for example, a user can connect the system to other apps on the phone, for example, Apple Health 401, Fitbit 402, Samsung Health 403, WHOOP 404, and Arise Fitness 405. The GUI is for example purposes, as one skilled in the art would understand other data and configurations of the display are possible.

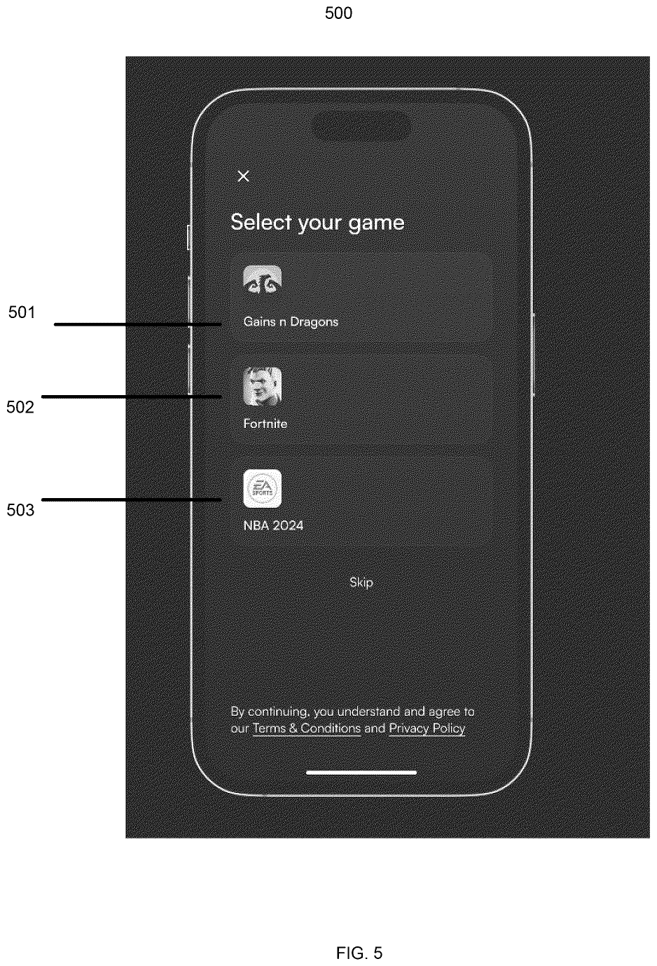

FIG. 5 is an example of a cell phone user application depicting an interface to initiate a data transfer to third party applications. This particular GUI 500 provides the user with the ability to set permissions and allow data transfer to various third party apps and third party businesses, for example, Gains n Dragons 501, Fortnite 502, NBA 503, etc. The GUI is for example purposes, as one skilled in the art would understand other data and configurations of the display are possible.

The GUIs illustrated in FIGS. 2-5 are by way of example. GUIs other than those depicted in FIGS. 2-5 are contemplated.

In addition, cell phone 110 can be utilized by the user to interact with the server 111 to securely log on, view, modify, and/or change certain data stored therein. Cell phone 110 can also be used to interact with server 111 to set privacy levels and/or permissions for the user's stored data and subsequent sharing of the user's data. In addition, a user can select a specific skills level that will be used by the server 111 in further processing. In addition to server 111 access through the cell phone 110, the system can also include user access through a web browser 140 to provide the user access to the user's data and account information and settings.

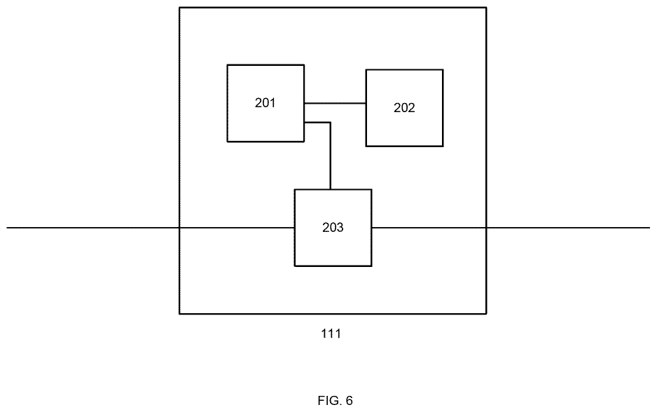

As shown in FIG. 6, server 111 contains at least one processor 201 and at least one data storage device 202. The server 111 receives the basic information and the bio data from the user and fitness data from the input devices and stores the basic information, the bio data, and the fitness data in the storage device 202. In addition, server 111 includes an interface device 203 for receiving and transmitting data to and from the server 111.

The server 111 collects and/or determines aggregated data based on the basic information, the bio data, fitness data, and user entered information. The bio data can be used to create thresholds for further calculations. The aggregated data can include body mass index (BMI), body fat, fat-free body weight, subcutaneous fat, visceral fat, muscle mass, skeletal muscle, body water, metabolic age, active calories burned, activity, steps, floors climbed, heart rate, sleep data, exercise data, nutrition data, weight and body composition, and mindfulness and stress. It is noted that the system calculates the data with the greatest accuracy when all aggregate data is provided, but can calculate less accurate aggregate data if some of the data is not available.

The aggregated data is received from different sources, for example, a smart scale, a smart watch, body sensors, smart weights, and the user itself. The body mass index (BMI), body fat, fat-free body weight, subcutaneous fat, visceral fat, muscle mass, skeletal muscle, body water, and metabolic age are some of the standard data generated by a smart scale. The active calories burned, activity, steps, floors climbed, heart rate, and sleep data are some of the standard data generated by a smart watch. Some aggregated data is user entered, for example, the exercise data is provided by the user and describes what type of exercise the user is engaged in (e.g., walking, running, swimming, etc.). This type of activity will affect the calculation of the associated skills data. Some data is entered directly by the user, for example, nutrition data. Some sensors detect body water content and body fat content used to determine weight and body composition. Still further some data is calculated from other raw data, for example mindfulness and stress are calculated from heart rate and respiration. It is noted that certain aggregated data may be produced by more than one input device, e.g., heart rate can be produced by a smart watch and a body sensor. In general, the aggregated data is sensor data obtained from sensors in the input devices, calculated from the sensor data, or entered by a user/third party.

Server 111 calculates skill data based on the fitness data, aggregated data, and other skills data. As an example, skill data can include speed, agility, strength, attack, defense, morale, discipline, etc. The skill data listed here correlates to skills found in popular fantasy adventure games. Server 111 is programmed to calculate this unique skill data using fitness data, aggregated data, and other skills data in varying levels.

In many computer games, eXperience Points (XP) are used to measure a player's experience and progression through the game. As experience points are gained, a character is described as leveling up to a more advanced skill level. As the skill level increases, the character becomes more powerful and able to complete more advanced objectives. In the present application, the data generated by the input devices or entered manually by the user is used to generate the skill data, which in turn is translated into XP.

The following table lists examples of the data used to determine the associated skills data:

TABLE 1

| | DATA USED TO DETERMINE |
|---|---|
| SKILL DATA | SKILL DATA |
| SPEED | DISTANCE, TIME |
| AGILITY | EXERCISE DATA, ACTIVITY, WEIGHT |
| | AND BODY COMPOSITION |
| STRENGTH | WEIGHT AND BODY COMPOSITION, |
| | MUSCLE MASS |
| ATTACK | STRENGTH, SPEED, AGILITY |
| DEFENSE | NUTRITION, SLEEP |
| MORALE | HEART RATE, MINDFULNESS, STRESS, |
| | SLEEP, BLOOD PRESSURE |
| DISCIPLINE | CONSISTENCY, STREAKS, ROUTINE |

### The Skill Data is Calculated Based on the Following Formulas:

### 1. Speed

$$100 XP ( experience points ) for each 1000 m you walk / run \qquad ( 1 )$$

It is noted that this can be adjusted for different distances. In addition, a standard speed calculation of distance×time can be used to calculate the speed skill data.

### 2. Agility

$$minutes of exercise * mix multiplier * second multiplier ( optional ) \qquad ( 2 )$$

where minutes of exercise is based on how much time the activity was performed, mix multiplier is how many different activities are performed, and the second multiplier (which is optional) is based on either BMI or body composition.

### BMI is Based on the Following Table Data:

TABLE 2

| | | MULTIPLIER |
|---|---|---|
| Underweight | <18.5 | 0.8 |
| Normal Weight | 18.5-−24.9 | 1 |
| Overweight | 25-29.9 | 0.8 |
| Obesity | >=30 | 0.6 |

### BODY COMPOSITION is Based on Table 3 (Male) and Table 4 (Female):

TABLE 3

| MALE BODY FAT | | MULTIPLIER |
|---|---|---|
| Essential Fat | 2-5% | 1.5 |
| Athletes | 6-13% | 1.4 |
| Fitness | 14-17% | 1.3 |
| Average | 18-24% | 1.2 |
| Overweight | 25-30% | 0.8 |
| Obese | >=31% | 0.6 |

TABLE 4

| FEMALE BODY FAT | | MULTIPLIER |
|---|---|---|
| Essential Fat | 10-13% | 1.5 |
| Athletes | 14-20% | 1.4 |
| Fitness | 21-24% | 1.3 |
| Average | 25-31% | 1.2 |
| Overweight | 32-39% | 0.8 |
| Obese | 40% and above | 0.6 |

### 3. Strength

calories spent*BMI or body composition multiplier*muscle mass multiplier(optional) (3)

where calories spent is equal to how many calories were burned during the fitness routine, the BMI or body composition multiplier is based on tabulated data (e.g., Table 5), and the muscle mass multiplier is read from Table 8 (Male) or Table 9 (Female).

### BMI is Based on the Following Table Data:

TABLE 5

| | | MULTIPLIER |
|---|---|---|
| Underweight | <18.5 | 0.8 |
| Normal Weight | 18.5-−24.9 | 1 |
| Overweight | 25-29.9 | 0.8 |
| Obesity | >=30 | 0.6 |

### BODY COMPOSITION is Based on Table 6 (Male) or Table 7 (Female) Following:

TABLE 6

| MALE BODY FAT | | MULTIPLIER |
|---|---|---|
| Essential Fat | 2-5% | 1.5 |
| Athletes | 6-13% | 1.4 |
| Fitness | 14-17% | 1.3 |
| Average | 18-24% | 1.2 |
| Overweight | 25-30% | 0.8 |
| Obese | >=31% | 0.6 |

TABLE 7

| FEMALE BODY FAT | | MULTIPLIER |
|---|---|---|
| Essential Fat | 10-13% | 1.5 |
| Athletes | 14-20% | 1.4 |
| Fitness | 21-24% | 1.3 |
| Average | 25-31% | 1.2 |
| Overweight | 32-39% | 0.8 |
| Obese | 40% and above | 0.6 |

### MUSCLE MASS MULTIPLIER is Based on Table 8 (Male) or Table 9 (Female) Following:

TABLE 8

| MALE | Low | Average | Ideal |
|---|---|---|---|
| MUSCLE | (multiplier | (multiplier | (multiplier |
| MASS | x0.8) | x1.0) | x1.2) |
| 18-35 | <36 | 36-40 | 40-44 |
| 36-55 | <32 | 32-36 | 36-40 |
| 56-75 | <28 | 28-32 | 32-35 |
| 76-85 | | | <31 |

TABLE 9

| FEMALE | Low | Average | Ideal |
|---|---|---|---|
| MUSCLE | (multiplier | (multiplier | (multiplier |
| MASS | x0.8) | x1.0) | x1.2) |
| 18-35 | <29 | 29-31 | 32-33 |
| 36-55 | <27 | 27-29 | 29-31 |
| 56-75 | <25 | 25-27 | 27-30 |
| 76-85 | | | <26 |

### 4. Attack

$$( strength XP + speed XP + agility XP ) / 3 \qquad ( 4 )$$

where strength XP, speed XP, and agility XP are previously calculated skill data.

### 5. Defense

$$hours slept and hours multiplier * kcal consumed and kcal multiplier ( optional ) \qquad ( 5 )$$

where kcal consumed and kcal multiplier are based on Tables 11 and 12 and hours slept is equal to

$$100 XP per hour slept * hours multiplier \qquad ( 6 )$$

where hours multiplier is determined based on Table 10.

TABLE 10

| | Recommended | Recommended | | Too Much | Too Little |
|---|---|---|---|---|---|
| | time (+−30 mins) | Interval | | Sleep/Day | Sleep/Day |
| AGE GROUP | (multiplier x1.2) | (multiplier x1.0) | | (multiplier x0.8) | (multiplier x0.8) |
| Infants | 14 hours | 12-16 | hours | >17 hours | <12 hours |
| (4-12 Months) | | | | | |
| Toddlers | 13 hours | 11-14 | hours | >15 hours | <11 hours |
| (1-2 Years) | | | | | |
| Preschool | 12 hours | 10-13 | hours | >14 hours | <10 hours |
| (3-5 Years) | | | | | |
| School-Age | 11 hours | 9-12 | hours | >13 hours | <9 hours |
| (6-12 Years) | | | | | |
| Teens | 9 hours | 8-10 | hours | >11 hours | <8 hours |
| (13-18 Years) | | | | | |
| Adults | 8 hours | 7-9 | hours | >10 hours | <7 hours |
| (18-64 Years) | | | | | |
| Older Adults | 7.5 hours | 7-8 | hours | >9 hours | <7 hours |
| (65+ Years) | | | | | |

KCAL CONSUMED AND KCAL MULTIPLIER are based on the following Tables 11 and 12:

TABLE 11

| Weight (Kg) | Men (Kcal/Day) | Women (Kcal/Day) |
|---|---|---|
| 40 | 1600 | 1400 |
| 45 | 1750 | 1500 |
| 50 | 1900 | 1600 |
| 55 | 2050 | 1700 |
| 60 | 2200 | 1800 |
| 65 | 2350 | 1900 |
| 70 | 2500 | 2000 |
| 75 | 2650 | 2100 |
| 80 | 2800 | 2200 |
| 85 | 2950 | 2300 |
| 90 | 3100 | 2400 |
| 95 | 3250 | 2500 |
| 100 | 3400 | 2600 |
| 105 | 3550 | 2700 |
| 110 | 3700 | 2800 |
| 115 | 3850 | 2900 |
| 120 | 4000 | 3000 |

TABLE 12

| Range Relative to | |
|---|---|
| Average Intake | MULTIPLIER |
| within ±5% | 1.5 |
| within ±10% | 1.2 |
| (excluding the 5% range) | |
| within ±15% | 1 |
| within ±20% | 0.8 |
| within ±25% | 0.6 |

### 6. Morale

$$heart rate * hours slept and hours multiplier \qquad ( 7 )$$

where heart rate is a rate measured by an input device and hours slept and hours multiplier are those previously defined above.
1 min with normal rate: 1×P

TABLE 13

| | | Heart Rate |
|---|---|---|
| Heart Rates | Age Group | (bpm) |
| Healthy Resting | Adults | 60-100 |
| Heart Rate | (18+ years) | bpm |
| | Children | 70-100 |
| | (6-15 years) | bpm |
| | Infants | 100-160 |
| | (0-12 months) | bpm |
| Tachycardia | Adults | Over 100 |
| (Too Fast) | (18+ years) | bpm |
| | Children | Over 120 |
| | (6-15 years) | bpm |
| | Infants | Over 160 |
| | (0-12 months) | bpm |
| Bradycardia | Adults | Below 60 |
| (Too Slow) | (18+ years) | bpm |
| | Children | Below 70 |
| | (6-15 years) | bpm |
| | Infants | Below 100 |
| | (0-12 months) | bpm |

### Hours Slept Multiplier

TABLE 14

| | Recommended | Recommended | | Too Much | Too Little |
|---|---|---|---|---|---|
| | (+−30 min) | Interval | | Sleep/Day | Sleep/Day |
| Age Group | (Multiplier x1.2) | (Multiplier x1.0) | | (Multiplier x0.8) | (Multiplier x0.8) |
| Infants | 14 | 12-16 | hours | >17 hours | <12 hours |
| (4-12 months) | | | | | |
| Toddlers | 13 | 11-14 | hours | >15 hours | <11 hours |
| (1-2 years) | | | | | |
| Preschool | 12 | 10-13 | hours | >14 hours | <10 hours |
| (3-5 years) | | | | | |
| School-age | 11 | 9-12 | hours | >13 hours | <9 hours |
| (6-12 years) | | | | | |
| Teens | 9 | 8-10 | hours | >11 hours | <8 hours |
| (13-18 years) | | | | | |
| Adults | 8 | 7-9 | hours | >10 hours | <7 hours |
| (18-64 years) | | | | | |
| Older Adults | 7.5 | 7-8 | hours | >9 hours | <7 hours |
| (65+ years) | | | | | |

### 7. Discipline

XP according to how many days in a row data is sent to the server by the user . . . (8) where the XP is read from Table 15, below.

TABLE 15

| Days in a Row | Total XP | Multiplier |
|---|---|---|
| 1 | 100 | 1.0 |
| 2 | 200 | 1.0 |
| 3 | 300 | 1.0 |
| 4 | 420 | 1.2 |
| 5 | 540 | 1.2 |
| 6 | 680 | 1.4 |
| 7 | 820 | 1.4 |
| 8 | 980 | 1.6 |
| 9 | 980 | 1.6 |
| 10 | 980 | 1.6 |
| 11 | 1160 | 1.8 |
| 12 | 1340 | 1.8 |
| 13 | 1520 | 1.8 |
| 14 | 1520 | 1.8 |

Now that the skill data has been determined, the skill data can be displayed (instantaneous and historically over time), personalized (e.g., based on historical data and user goals), and integrated with APIs for third party use (e.g., in an RPG game).

Finally, the server creates an endpoint for SDK integration, that is the XP is sent to the SDK.

The server 111 can then transmit the skills data to third party apps through an application programming interface (API). The skills data is utilized in a unique fashion by the third party apps. For example, if the third party app is a video game, the skills data can be used to adjust the experience points (XP) level of the user in the game.

The system integrates real-world fitness data into a gamified experience by converting health and activity metrics into role-playing game (RPG) attributes like Speed, Strength, Agility, Attack, Defense, Morale, and Discipline. This innovative approach bridges physical activity and virtual worlds, enabling users to enhance in-game experiences across diverse gaming genres. By leveraging data collected from platforms like Apple Health, Samsung Health, and Fitbit, the app generates XP (experience points) and skill levels that directly impact gameplay through an API integration with Unity SDK. Developers can customize gameplay mechanics, unlock unique content, and improve player engagement by linking fitness achievements to gaming rewards.

The following is a description of some of the applications of the skill data across various gaming genres.
1. Racing Games: Real-life fitness data translates into enhanced vehicle performance and exclusive rewards. Players can unlock faster speeds, better acceleration, and unique car skins based on their physical activity.
Speed: Increases the ca's top speed.
Strength: Boosts acceleration for quicker starts.
Agility: Enhances cornering and stability.
Attack: Improves ramming damage or offensive tools.
Defense: Reduces car damage in collisions.
Morale: Enhances nitro or turbo system recovery.
Discipline: Unlocks exclusive car skins and upgrades.
2. Action/Adventure Games: Physical fitness levels enhance movement, combat, and exploration mechanics. Players experience faster travel, stronger melee attacks, and access to rare rewards.
Speed: Improves running and movement speed.
Strength: Enables powerful melee attacks and heavy object interactions.
Agility: Increases dodge, climb, and jump capabilities.
Attack: Enhances weapon effectiveness and combos.
Defense: Adds resistance to damage or increases health regeneration.
Morale: Buffs stamina and skill cooldown reductions.
Discipline: Unlocks story-based rewards and rare items.
3. Sports Games: Fitness achievements boost athletic performance, precision, and teamwork dynamics, with the added reward of unique in-game gear.
Speed: Boosts sprint and reaction times.
Strength: Enhances power shots and tackles.
Agility: Improves turning, dribbling, or dodging.
Attack: Adds precision to scoring opportunities.
Defense: Strengthens blocking or goalkeeping skills.
Morale: Improves momentum for team plays.
Discipline: Rewards unique gear and better team coordination.
4. RPGs (Role-Playing Games): Fitness data enriches character stats and unlockables, driving engagement through progress tied to real-world effort.
Speed: Increases travel and dodge speed.
Strength: Improves melee damage and heavy weapon handling.
Agility: Buffs stealth, lock-picking, and evasion.
Attack: Enhances overall offensive prowess.
Defense: Adds resistance to damage types.
Morale: Boosts critical hit chances and stamina.
Discipline: Unlocks rare spells, titles, or mounts.
5. Shooter Games: Improved tactical performance and customization come from real-world activities, fostering competitive advantages.
Speed: Quickens sprint and reload speeds.
Strength: Reduces recoil and improves melee power.
Agility: Boosts accuracy during rapid movement.
Attack: Enhances weapon power and bullet effects.
Defense: Increases shield durability or health regeneration.
Morale: Adds adrenaline boosts for faster recovery.
Discipline: Unlocks exclusive weapon skins or perks.
6. Fighting Games: Combos, evasion, and power attacks are enhanced by fitness stats, offering a more dynamic combat experience.
Speed: Quickens attack and movement speeds.
Strength: Increases damage dealt by physical attacks.
Agility: Enhances combo execution and evasion.
Attack: Buffs offensive abilities and special moves.
Defense: Reduces damage taken and increases blocking efficiency.
Morale: Activates recovery boosts during critical fights.
Discipline: Rewards unique costumes and special effects.
7. Simulation Games: Real-world productivity and adaptability are mirrored in-game, offering streamlined efficiency and creative unlocks.
Speed: Accelerates character movements or production rates.
Strength: Boosts productivity in physical tasks.
Agility: Improves multitasking abilities or precision.
Attack: Adds effectiveness in challenging scenarios.
Defense: Ensures system stability under duress.
Morale: Keeps characters or workers motivated.
Discipline: Unlocks advanced features or efficiencies.
8. Puzzle Games: Fitness inputs improve problem-solving dynamics and unlock aesthetic upgrades.
Speed: Reduces puzzle completion time.
Strength: Enables solving power-based challenges.
Agility: Improves adaptability to dynamic puzzles.
Attack: Enhances special move potency (e.g., power-ups).
Defense: Adds hints or buffer zones for mistakes.
Morale: Reduces penalties for errors.
Discipline: Unlocks harder levels or aesthetic upgrades.
9. Strategy Games: Tactical flexibility and fortification strength are bolstered by fitness data, adding depth to strategic planning.
Speed: Reduces cooldown times or boosts movement rates.
Strength: Adds power to units or structures.
Agility: Enhances tactical flexibility.
Attack: Increases effectiveness of offensive units.
Defense: Boosts fortification strength and resilience.
Morale: Improves unit efficiency or recovery.
Discipline: Unlocks advanced technologies or units.
10. MOBA Games: Hero attributes and synergy improve as fitness translates into better precision, durability, and exclusive content.
Speed: Increases movement and cooldown reduction.
Strength: Buffs hero damage and durability.
Agility: Improves targeting and skill precision.
Attack: Enhances critical strikes or ability damage.
Defense: Adds shields or health regen effects.
Morale: Improves team synergy and comeback potential.
Discipline: Unlocks skins, emotes, or unique abilities.
11. Survival Games: Adaptability and endurance mechanics are influenced by user fitness levels, elevating resource management and exploration.
Speed: Speeds up resource gathering or movement.
Strength: Increases carrying capacity or damage.
Agility: Improves crafting efficiency or stealth.
Attack: Boosts hunting or fighting capabilities.
Defense: Increases resistance to hazards or hunger.
Morale: Keeps characters healthy under stress.
Discipline: Unlocks better tools and survival recipes.
12. Horror Games: Players gain survival advantages, enabling better escapes and resilience against in-game stressors.
Speed: Improves escape ability.
Strength: Boosts interactions like barricading doors.
Agility: Enhances stealth and evasion.
Attack: Increases effectiveness of self-defense actions.
Defense: Adds resistance to scares or injuries.
Morale: Maintains composure in high-stress moments.
Discipline: Rewards unique endings or collectibles.
13. Platformers: Fitness stats impact traversal and exploration, with added benefits like unlocking secret areas.
Speed: Improves running and jumping distances.
Strength: Increases breakable obstacle interactions.
Agility: Boosts mid-air control and precision.
Attack: Adds power to offensive actions.
Defense: Reduces fall damage or collision penalties.
Morale: Activates buffs after streak successes.
Discipline: Unlocks secret levels or characters.
14. Sandbox Games: Fitness data encourages creativity and exploration, with enhanced resource-gathering capabilities.
Speed: Boosts movement across large maps.
Strength: Improves crafting and building efficiency.
Agility: Enhances adaptability in dynamic environments.
Attack: Improves tools for resource gathering.
Defense: Increases environmental hazard resistance.
Morale: Activates creativity boosts in building modes.
Discipline: Unlocks rare items and resources.
15. MMORPGs (Massively Multiplayer Online Role-Playing Games): Fitness-driven character progression supports long-term engagement and unique multiplayer rewards.
Speed: Increases movement and mount speeds.
Strength: Boosts melee damage and physical resistance.
Agility: Improves dodging and resource-gathering rates.
Attack: Enhances DPS (damage per second) effectiveness.
Defense: Strengthens armor or shield durability.
Morale: Activates buffs during long battles or raids.
Discipline: Unlocks exclusive guild rewards or unique titles.
16. Battle Royale Games: Survival, precision, and speed are directly influenced by fitness metrics, elevating player customization and competitiveness.
Speed: Improves sprinting and looting speeds.
Strength: Reduces fall damage or enhances melee power.
Agility: Enhances quick movement and accurate shooting.
Attack: Buffs damage for ranged and close-combat weapons.
Defense: Adds temporary shields or healing boosts.
Morale: Speeds up recovery after intense fights.
Discipline: Unlocks rare skins, parachutes, or emotes.
17. Rhythm Games: Players enjoy enhanced reaction times and accuracy, rewarded with exclusive content for consistent performance.
Speed: Improves reaction time to fast-paced notes.
Strength: Adds power to long-note holds or stamina-based songs.
Agility: Increases accuracy during complex note patterns.
Attack: Boosts score multipliers for consecutive streaks.
Defense: Provides leniency during missed notes.
Morale: Enhances streak consistency after successes.
Discipline: Unlocks exclusive songs or visual effects.
18. Open World Games: Real-life fitness encourages immersive exploration and interaction, unlocking hidden locations and rewards.
Speed: Improves map traversal and exploration efficiency.
Strength: Enhances interactions with destructible environments.
Agility: Boosts parkour or climbing mechanics.
Attack: Adds damage to weapons and tools.
Defense: Improves resistance to environmental damage.
Morale: Increases rewards for completing exploration streaks.
Discipline: Unlocks hidden locations and rewards.
19. Card Games: Strategy and precision are boosted through fitness achievements, with rare cards as additional incentives.
Speed: Reduces cooldown between moves or rounds.
Strength: Buffs card abilities that deal damage.
Agility: Enhances tactical flexibility for using combos.
Attack: Improves offensive card effectiveness.
Defense: Adds protective bonuses to defensive cards.
Morale: Activates passive buffs during win streaks.
Discipline: Unlocks rare cards or exclusive decks.
20. Tower Defense Games: Tower strength and resource efficiency are amplified through physical activity, providing access to advanced features.
Speed: Speeds up resource collection or tower building.
Strength: Increases tower damage or health.
Agility: Enhances tower targeting speed and accuracy.
Attack: Boosts overall firepower of defenses.
Defense: Adds shields or resistance to towers.
Morale: Improves resource regeneration during long waves.
Discipline: Unlocks advanced towers or abilities.
21. Stealth Games: Real-world fitness levels improve evasion and stealth mechanics, offering enhanced gameplay routes and gadgets.
Speed: Improves movement without detection.
Strength: Enhances takedown efficiency and stealth kills.
Agility: Increases ability to bypass traps or security.
Attack: Adds precision to stealth-based weapons.
Defense: Reduces detection penalties.
Morale: Activates stress resistance under pursuit.
Discipline: Unlocks hidden routes or gadgets.
22. Party Games: Quick reflexes and coordination, driven by fitness data, heighten minigame competitiveness and rewards.
Speed: Boosts reaction times in mini-games.
Strength: Adds bonuses to physical or timing challenges.
Agility: Improves performance in complex mini-games.
Attack: Enhances competitiveness in player-versus-player games.
Defense: Adds error tolerance in fast-paced events.
Morale: Activates bonuses for team games.
Discipline: Unlocks special mini-games or characters.
23. Roguelike/Roguelite Games: Fitness accomplishments translate into better survival rates and rarer loot acquisition.
Speed: Increases movement speed between dungeon levels.
Strength: Enhances weapon and skill damage.
Agility: Improves dodging and evasion.
Attack: Adds critical strike potential.
Defense: Buffs resistance to damage types.
Morale: Provides bonuses for streaks of successful runs.
Discipline: Unlocks rare items or higher difficulties.
24. Idle Games: Passive resource generation is sped up, with added rewards for regular activity.
Speed: Speeds up passive resource accumulation.
Strength: Boosts production rates of critical resources.
Agility: Increases flexibility in auto-management systems.
Attack: Adds potency to automated offensive mechanics.
Defense: Improves durability of structures or systems.
Morale: Enhances long-term rewards during active play.
Discipline: Unlocks faster progression and rare items.
25. Puzzle/Adventure Games: Fitness engagement makes intricate puzzles easier to solve, offering bonus content for dedicated players.
Speed: Reduces time spent solving intricate puzzles.
Strength: Enables interaction with heavy puzzle elements.
Agility: Improves responsiveness during timed puzzles.
Attack: Adds offensive options to puzzle-solving.
Defense: Provides safeguards against penalties.
Morale: Maintains consistency during puzzle streaks.
Discipline: Unlocks bonus challenges or collectibles.

The server 111 can also transmit the skills data to third party businesses. The skills data is utilized in a unique fashion by the third party businesses. For example, if the third party business is a health insurance provider, the user can be afforded a discount based on the user's level of skill data. In another example, the skills data can be used to provide discounts on products such as fitness training equipment or apparel through third party retailers. Some other examples of third party businesses include health care providers and fitness centers, each being capable of providing user specific discounts to the user based on the user's skills data.

The server 111 also includes an equalizing algorithm that adjusts skills data should some aggregated data not be available for a specific user. For example, if an input device for user A only provides step data and active calories burned, and an input device for user B provides all of the aggregated data, server 111 will utilize the equalizing algorithm to equalize the skills data so that user A and user B have weighted adjustments to their XP. This equalization can be based on large language modeling (LLM) or driven by third party apps/businesses, i.e., the skills data can be customized to fit the needs of the third party apps or businesses.

A method for motivating and driving fitness and exercise routines is also disclosed herein.

In the method, in step 701 the server receives bio data including at least one of the user's age, gender, height, and weight from a user and stores the bio data in the memory. The user can utilize the cell phone app or the web browser app to enter the bio data into the system.

In step 702, the server receives aggregated data from the various input devices and stores the aggregated data in the memory. As stated earlier, the aggregated data can be received from various types of devices, for example, smart watches, smart scales, smart weights, sensors worn be the user, or even user entered through smart watch, call phone app or web browser app, etc.

In step 703 the server receives at least one of vital sign data and movement data obtained from the fitness devices during an exercise routine of the user and stores the at least one of the vital sign data and the movement data in the memory.

In step 704 the server reads the aggregated data, the vital sign data and the movement data from the memory and calculates skills data based on the aggregated data, the vital sign data and the movement data.

In step 705 the server customizes the skill data for a particular third party app or business. This customization can take the form of a rating that can be used by a business to provide the user with a discount, or add experience points (XP) to a user in a video game.

In step 706, the server transmits the customized skills data to the third party apps or businesses. This process can include the generation by the system of a character that is ported over into the third party app or the system can port over the skills data and the third party app would use the skills data to enhance the in-app character.

As stated above, there are situations where the skill data of different users will be based on incomplete aggregated data. In this situation an equalization algorithm is required to ensure that end use is balanced across all of the users when applied to a third party app or by a third party business.

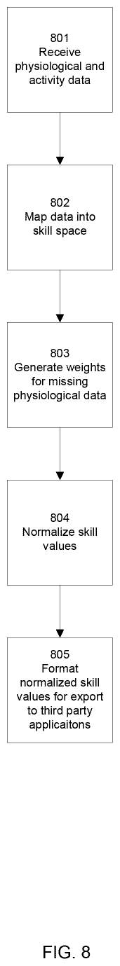

FIG. 8 is a flowchart illustrating the equalization and normalization processes according to the present disclosure. In step 801 the system receives physiological data and activity data from the fitness devices. In step 802, the normalization engine maps the data into a canonical multi-dimensional skill space. In step 803 the missing data compensation engine infers or weights absent physiological parameters based on available parameters and historical user models. In step 804, the fairness equalization engine normalizes the resulting skill values across users having different sensor coverage. Finally, and optionally, the API layer can export the normalized skill values to third party applications independently of the original biometric sources.

The system and method disclosed herein solves the problem of having a lack of motivation during an exercise routine by enhancing a user experience in an interactive application (app) based on previous user fitness and exercise routines.

All referenced publications are incorporated by reference herein in their entirety for the purpose of describing and disclosing, for example, materials, constructs, and methodologies that can be used in connection with the presently described invention.

Included in the description are flowcharts depicting examples of the methodology which may be used to conduct a goal-directed semantic search. In the following description, it will be understood that each block of the flowchart illustrations, and combinations of blocks in the flowchart illustrations, can be implemented by computer program instructions. These computer program instructions may be loaded onto a computer or other programmable apparatus to produce a machine such that the instructions that execute on the computer or other programmable apparatus create means for implementing the functions specified in the flowchart block or blocks. These computer program instructions may also be stored in a computer-readable memory that can direct a computer or other programmable apparatus to function in a particular manner such that the instructions stored in the computer-readable memory produce an article of manufacture including instruction means that implement the function specified in the flowchart block or blocks. The computer program instructions may also be loaded onto a computer or other programmable apparatus to cause a series of operational steps to be performed in the computer or on the other programmable apparatus to produce a computer implemented process such that the instructions that execute on the computer or other programmable apparatus provide steps for implementing the functions specified in the flowchart block or blocks.

Accordingly, blocks of the flowchart illustrations support combinations of means for performing the specified functions and combinations of steps for performing the specified functions. It will also be understood that each block of the flowchart illustrations, and combinations of blocks in the flowchart illustrations, can be implemented by special purpose hardware-based computer systems that perform the specified functions or steps, or combinations of special purpose hardware and computer instructions.

Some portions of this specification may be presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve the manipulation of information elements. Typically, but not necessarily, such elements may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” “words”, or the like. These specific words, however, are merely convenient labels and are to be associated with appropriate information elements.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As will be understood by those familiar with the art, the invention may be embodied in other specific forms without departing from the spirit or essential characteristics thereof. Likewise, the particular naming and division of the modules, managers, functions, systems, engines, layers, features, attributes, methodologies, and other aspects are not mandatory or significant, and the mechanisms that implement the invention or its features may have different names, divisions, and/or formats. Furthermore, as will be apparent to one of ordinary skill in the relevant art, the modules, managers, functions, systems, engines, layers, features, attributes, methodologies, and other aspects of the invention can be implemented as software, hardware, firmware, or any combination of the three. Of course, wherever a component of the present invention is implemented as software, the component can be implemented as a script, as a standalone program, as part of a larger program, as a plurality of separate scripts and/or programs, as a statically or dynamically linked library, as a kernel loadable module, as a device driver, and/or in every and any other way known now or in the future to those of skill in the art of computer programming. Additionally, the present invention is in no way limited to implementation in any specific programming language, or for any specific operating system or environment. Accordingly, the disclosure of the present invention is intended to be illustrative, but not limiting, of the scope of the invention, which will be set forth in the forthcoming claims. Various APIs can be utilized where needed to connect the system to other third-party apps and systems.

The present teachings encompass embodiments in other specific forms without departing from the spirit or essential characteristics thereof. The foregoing embodiments are therefore to be considered illustrative in all respects rather than limiting on the present teachings described herein. The scope of the present invention is thus indicated by the appended claims rather than by the foregoing description, and all changes that come within the meaning and range of equivalency of the claims are intended to be embraced therein.

What is claimed is:

1. A system for motivating exercise routines, comprising:
at least one server communicatively coupled to,
at least one data storage device configured to store at least one of bio data, vital sign data, movement data, fitness data, aggregated data, and skills data; and
at least one fitness device utilized by a user to generate at least one of the vital sign data, the movement data, and the aggregated data produced by the user and communicates the at least one of the vital sign data, the movement data, and the aggregated data to the server,
wherein the at least one server executes a method, comprising:
receiving the bio data from the user;
storing the bio data in the data storage device;
receiving the at least one of the vital sign data, the movement data, and the aggregated data from the fitness device;
storing the at least one of vital sign data, the movement data, and the aggregated data in the storage device; and
reading the at least one of vital sign data, the movement data, and the aggregated data from the storage device;
generating the skills data based on the at least one of vital sign data, the movement data, and the aggregated data;
weighting the skills data based on available parameters and historical user models;
normalizing the weighted skills data across multiple users.

2. The system of claim 1, wherein the fitness device includes a smart device used by the user during an exercise routing to generate the movement data regarding the movement of the user or the vital sign data of the user.

3. The system of claim 1, wherein,
the bio data includes at least one of age, gender, height, and weight of the user;
the fitness data includes at least one of vital sign data and movement data of the user;
the aggregated data includes at least one of body mass index (BMI), body fat, fat-free body weight, subcutaneous fat, visceral fat, muscle mass, skeletal muscle, body water, metabolic age, active calories burned, activity, steps, floors climbed, heart rate, sleep data, exercise data, nutrition data, weight and body composition, and mindfulness and stress; and,
the skills data includes at least one of speed, agility, strength, attack, defense, morale, and discipline.

4. The system of claim 1, wherein the fitness device includes an accelerometer to generate at least one of speed, acceleration and direction of the fitness device obtained from movement of the fitness device.

5. The system of claim 1, wherein the fitness device includes a heart rate monitor to generate heart rate data of the user.

6. The system of claim 1, wherein the skill data is transmitted to a third-party application.

7. The system of claim 1, wherein
the skills data is used to enhance a character in the system; and
the character is ported into a third-party application.

8. The system of claim 1, wherein the skills data is converted into experience Points (XP) used to measure a user's experience and progression through a game the user is engaged in.

9. The system of claim 3, wherein:
speed is calculated based on distance and time of a user;
agility is calculated based on exercise data, activity, and weight and body composition;
strength is calculated based on weight and body composition, and muscle mass;
attack is calculated based on strength, speed, and agility;
defense is calculated based on nutrition, and sleep;
morale is calculated based on, heart rate, mindfulness, stress, sleep, and blood pressure; and
discipline is calculated based on consistency, streaks, and routine.

10. The system of claim 1, wherein the aggregated data is received from at least one of a smart scale, a smart watch, body sensors, and smart weights.

11. A method for motivating and driving fitness and exercise routines, comprising the steps of:
receiving the bio data from a user;
storing the bio data in the data storage device;
receiving the at least one of vital sign data, movement data, and aggregated data from a fitness device;
storing the at least one of vital sign data, the movement data, and the aggregated data in the storage device;
reading the at least one of vital sign data, the movement data, and the aggregated data from the storage device; and
generating the skills data based on the at least one of vital sign data, the movement data, and the aggregated data.

12. The method of claim 11, wherein,
the bio data includes at least one of age, gender, height, and weight of the user;
the aggregated data includes at least one of body mass index (BMI), body fat, fat-free body weight, subcutaneous fat, visceral fat, muscle mass, skeletal muscle, body water, metabolic age, active calories burned, activity, steps, floors climbed, heart rate, sleep data, exercise data, nutrition data, weight and body composition, and mindfulness and stress; and,
the skills data includes at least one of speed, agility, strength, attack, defense, morale, and discipline.

13. The method of claim 12, wherein the skill data is transmitted to a third-party application.

14. The method of claim 12, wherein
the skills data is used to enhance a character in the system; and
the character is ported into a third-party application.

15. The method of claim 11, wherein the vital sign data and movement data is received from a fitness device that includes a smart device used by the user during an exercise routing to generate the movement data regarding the movement of the user or the vital sign data of the user.

16. The method of claim 12, wherein:
speed is calculated based on distance and time of a user;
agility is calculated based on exercise data, activity, and weight and body composition;
strength is calculated based on weight and body composition, and muscle mass;
attack is calculated based on strength, speed, and agility;
defense is calculated based on nutrition, and sleep;
morale is calculated based on, heart rate, mindfulness, stress, sleep, and blood pressure; and
discipline is calculated based on consistency, streaks, and routine.

17. A non-transitory computer-readable recording medium storing a program to execute a method for motivating and driving fitness and exercise routines, the method comprising the steps of:
receiving the bio data from a user;
storing the bio data in the data storage device;
receiving the at least one of vital sign data, movement data, and aggregated data from a fitness device;
storing the at least one of vital sign data, the movement data, and the aggregated data in the storage device;
reading the at least one of vital sign data, the movement data, and the aggregated data from the storage device; and
generating the skills data based on the at least one of vital sign data, the movement data, and the aggregated data.

18. The method of claim 17, wherein,
the bio data includes at least one of age, gender, height, and weight of the user;
the aggregated data includes at least one of body mass index (BMI), body fat, fat-free body weight, subcutaneous fat, visceral fat, muscle mass, skeletal muscle, body water, metabolic age, active calories burned, activity, steps, floors climbed, heart rate, sleep data, exercise data, nutrition data, weight and body composition, and mindfulness and stress; and,
the skills data includes at least one of speed, agility, strength, attack, defense, morale, and discipline.

19. The method of claim 18, wherein the skill data is transmitted to a third-party application.

20. The method of claim 18, wherein
the skills data is used to enhance a character in the system; and
the character is ported into a third-party application.

21. A system for generating game ready skill values from heterogeneous biometric sources, comprising:
a server configured to receive multiple physiological and activity datasets from heterogeneous fitness devices;
a normalization engine that maps those datasets into a canonical multi-dimensional skill space;
a missing data compensation engine that infers or weights absent physiological parameters based on available parameters and historical user models;
a fairness equalization engine that normalizes the resulting skill values across users having different sensor coverage; and
an API layer that exposes the normalized skill values to third party applications independently of the original biometric sources.
